(19) United States
(12) Patent Application Publication
(10) Pub. No.: US 2016/0143861 A1
(43) Pub. Date: May 26, 2016

(54) COMPOSITIONS COMPRISING HYDROXYTYROSOL, RESVERATROL, LYCOPENE, FLAVANOLS, AND/OR FLAVONOIDS AND USE THEREOF

(71) Applicant: SPECIALTY NUTRITION GROUP, INC., Lighthouse Point, FL (US)

(72) Inventor: Gregory T. Hom, Lighthouse Point, FL (US)

(73) Assignee: Specialty Nutrition Group, Inc., Lighthouse Point, FL (US)

(21) Appl. No.: 14/904,403

(22) PCT Filed: Jul. 11, 2014
(86) PCT No.: PCT/US2014/046287

§ 371 Date: Jan. 11, 2016

Related U.S. Application Data
(60) Provisional application No. 61/845,284, filed on Jul. 11, 2013.

(51) Int. Cl. A61K 31/05; A61K 31/353; A61K 36/53; A61K 36/87; A61K 36/899; A61K 36/23; A61K 36/81; A61K 36/21; A61K 36/42; A61K 36/88; A61K 31/01; A61K 36/31

(52) U.S. Cl. CPC A61K 31/05; A61K 31/01; A61K 31/353; A61K 36/53; A61K 36/87; A61K 36/31; A61K 36/23; A61K 36/81; A61K 36/21; A61K 36/42; A61K 36/88; A61K 36/899

(57) ABSTRACT

The present invention refers to novel compositions comprising hydroxytyrosol, resveratrol, lycopene, flavanols, and/or flavonoids and their use in treating and/or preventing pro-inflammatory diseases or disorders and ROS-mediated diseases or disorders. This invention also generally relates to compositions and to methods of using the compositions to increase tissue oxygenation.

### CROSS-REFERENCE TO RELATED APPLICATION

This application claims the benefit of and priority to U.S. Provisional Patent Application Ser. No. 61/845,284, filed Jul. 11, 2013. The entire contents of this patent application are hereby incorporated by reference herein.

### BACKGROUND OF THE INVENTION

In a healthy cell, the level of reactive oxygen species (ROS) is tightly regulated by the antioxidant defense system. However, upon environmental stress or cellular damage, the cell cannot readily detoxify the ROS generated and may thereby suffer from oxidative stress, which is implicated in the pathogenesis of many age-related diseases, such as inflammation, cancer, Alzheimer's disease and other neurodegenerative disorders, stroke, chronic kidney disease, type II diabetes, cardiovascular disease, and aging itself [Liu, Y.; Kern, J. T.; Walker, J. R.; Johnson, J. A.; Schultz, P. G.; Luesch, H. A genomic screen for activators of the antioxidant response element. Proc Natl Acad Sci USA. 104:5205-10; 2007; Dinkova-Kostova A T, Massiah M A, Bozak R E, Hicks R J, Talalay P. Potency of Michael reaction acceptors as inducers of enzymes that protect against carcinogenesis depends on their reactivity with sulfhydryl groups. Proc Natl Acad Sci USA 2001; 98:3404-3409; Dinkova-Kostova, A T, Liby K T, Stephenson K K, Holtzclaw W D, Gao X, Suh N et. al. Extremely potent triterpenoid inducers of the phase 2 response: Correlations of protection against oxidant and inflammatory stress. Proc Natl Acad Sci USA 2005; 102:4584-4589; Pergola P E, Raskin P, Toto R D, Meyer C J, Huff J W, Grossman E B et. al. BEAM Study Investigators. Bardoxolone methyl and kidney function in CKD with type 2 diabetes. N Engl J Med 2011; 365:327-336].

In addition to the established research linking oxidative stress to inflammation, diabetes, and diseases of aging (e.g., cardiovascular disease, neurodegenerative disorders, etc.), recent data suggest that inflammation itself plays a major role in the onset and progression of these same diseases. Furthermore, research has shown that arresting the inflammatory component associated with these diseases often results in significant improvement in managing, ameliorating, and/or treating these diseases and, thus, anti-inflammatory therapy is suggested to be an attractive approach for treating diseases such as aging and aging-related diseases, inflammation, (e.g., rheumatoid arthritis, skin inflammation, and osteoarthritis), cardiovascular disease (e.g., dyslipidemia, athlerosclerosis, congestive heart failure, hypertension, etc.), diabetes, pre-diabetes, obesity, cognition disorders, hypercholesterolemia, pain disorders associated with inflammation, thyroid disorders (e.g., hyperthyroidism, hypothyroidism), allergic reactions, and improvements in soft-tissue performance (e.g., oxygenation).

It is also widely accepted that increased oxygenation to tissues (e.g., muscle and skin) improves the performance, function, and/or appearance of said tissues. Non-limiting examples include: a) increasing blood hemoglobin oxygen saturation (e.g., SO2, SaO2); b) decreasing partial pressure of oxygen (SpO2); c) decreasing transcutaneous pressure of oxygen; d) improving VO2 measurements (i.e., measurement of the utilization efficiency of oxygen); and e) increasing oxygen saturation in the microcirculation (StO2). This relationship between tissue oxygenation and performance has been thoroughly demonstrated in the field of athletics (e.g., increased blood hemoglobin oxygen saturation increases athletic strength and endurance).

Cardiovascular disease is one of the leading causes of morbidity and mortality. Historically, identification of cardiovascular disease risk and treatment usually focus on management of blood lipids. Although it is known that inflammation may occur in conjunction with cardiovascular disease even in very early stages of the disease, management of the inflammatory response is not often a priority and, when it is, therapy specifically targeting the inflammatory component often comes following a cardiovascular event. Therefore, one aspect of this invention is anti-inflammatory intervention prior to a cardiovascular event with any of the compounds or compositions delineated herein, or combinations thereof. Although the involvement of the inflammatory processes in cardiovascular disease is not completely understood, statins are known to have an anti-inflammatory action and there is a growing view that their benefits may in part be related to this anti-inflammatory activity. In addition to cardiovascular disease, research has also shown positive impact of affecting the inflammatory and oxidative-stress components in treating other diseases with metabolic components, such as, but not limited to, diabetes, hypercholesterolemia (e.g., elevated low-density lipoprotein (LDL) cholesterol, elevated total cholesterol, low high-density lipoprotein (HDL) cholesterol), metabolic syndrome, and obesity. [European Heart Journal Supplements (2002) (Supplement A), A18-A25, 1520-765X/02/0A0018+08 J. Nilsson, M. P. S. Ares, M. Lindholm, G. Nordin Fredrikson and S. Jovinge; MMW Fortschr Med. 2004 Sep 2; 146(35-36):32-3, 35-6; Löbner K, Füchtenbusch M; Diabetes (2004) Low-Grade Systemic Inflammation and the Development of Type 2 Diabetes: The Atherosclerosis Risk in Communities Study, Bruce B. Duncan, Maria Inês Schmidt, James S. Pankow, Christie M. Ballantyne, David Couper, Alvaro Vigo, Ron Hoogeveen, Aaron R. Folsom and Gerardo Heiss; Nature Medicine 11, 191-198 (2005) Published online: 30 January 2005, KK-β links inflammation to obesity-induced insulin resistance Melek C Arkan, Andrea L Hevener, Florian R Greten, Shin Maeda, Zhi-Wei Li Jeffrey M Long, Anthony Wynshaw-Boris, Giuseppe Poli, Jerrold Olefsky & Michael Karin; Current Diabetes Reports 2005, Volume 5, Issue 1, pp 70-75 The evolving role of inflammation in obesity and the metabolic syndrome, Yong-Ho Lee PhD, Richard E. Pratley MD; Mediators of Inflammation Volume 2010 (2010), Chronic Inflammation in Obesity and the Metabolic Syndrome, Rosário Monteiro and Isabel Azeveda].

Sun exposure, and particularly exposure to ultraviolet wavelengths of sunlight, causes damage to the skin, resulting in photoaging (cosmetic damage to skin) as well as medical conditions such as sunburn, and, in some cases, skin cancers. Current methods for protecting against damage from sunlight include chemical sunscreens. While these compounds can be effective in reducing exposure to damaging sunlight, some compounds are suspected of acting as endocrine disruptors when absorbed through the skin. Also, chemical sunscreens do not protect against all forms of photoaging caused by sun exposure. Cost and convenience, including the need for frequent topical re-application, further limit the effectiveness of chemical topical sunscreens. Physical sunscreens, such as zinc oxide and titanium dioxide, also can be effective sunblocks, but topical application and cosmetic appearance of these sunblocks can limit their acceptance.

Certain oral supplements have been reported to provide protection from sunburn. For example, astaxanthin, a carotenoid pigment, has been reportedly used for sun protection, see, e.g., U.S. Pat. No. 6,433,025; the mechanism of action is believed to be at least in part due to the antioxidant properties of astaxanthin. Extracts of ferns of the genus Polypodium have been reported to be photoprotective, as discussed in U.S. Pat. No. 5,614,197, possibly due to an immunomodulatory effect. The use of antioxidants and other nutritional substances to retard photoaging and its mechanisms (primarily through MMP (matrix metalloproteinase) inhibition for sun protection are discussed in U.S. Patent Application 20050058709.

Mammals, especially humans, can be afflicted with acute and chronic conditions that result in pain and inflammation. Examples of such conditions include: osteoarthritis, rheumatoid arthritis, soft tissue injury as well as exercise-related conditions e.g. sprain, exertion related muscle soreness, back strain, tendonitis. Reduction in pain may be accomplished by controlling the inflammatory process at or following onset. In addition, insuring that the inflammatory process is well-regulated prior to an initiating event may reduce pain and shorten the time required to return to a healthier state. In conditions like osteoarthritis, optimal control of pain and inflammation is essential in improving range of motion and restoring functional ability.

However, no single therapy exists that treats and/or prevents the aforementioned diseases or disorders by specifically targeting the inflammatory and oxidative-stress components for inflammatory- and ROS-mediated diseases or disorders using naturally-derived components. There is an extensive and growing body of literature describing the cardiovascular and metabolic benefits of following a Mediterranean diet. Such diets are rich in foods like olives, tomatoes, and wine that contain bioactive compounds. Consuming the optimal spectrum and amounts of bioactives on a regular basis is not easy and, therefore, a specially designed composition is of value. Therefore, additional compositions comprising such bioactive compounds and methods for the treatment and/or prevention of aging and aging-related diseases, inflammation, (e.g., rheumatoid arthritis, skin inflammation, and osteoarthritis), cardiovascular disease (e.g., dyslipidemia, athlerosclerosis, congestive heart failure, hypertension, etc.), diabetes, pre-diabetes, obesity, cognition disorders, hypercholesterolemia, pain disorders associated with inflammation, thyroid disorders, allergic reactions, improvements in soft-tissue performance (e.g., oxygenation), diseases mediated through ROS, hypercholesterolemia (e.g., elevated low-density lipoprotein (LDL) cholesterol, elevated total cholesterol, low high-density lipoprotein (HDL) cholesterol), metabolic syndrome, obesity, photoaging, sunburn, and other forms of damage resulting from exposure to ultraviolet radiation would be desirable.

### COMPOUNDS OF THE INVENTION

The invention relates generally to compositions and to methods of using the compositions to treat and/or prevent pro-inflammatory diseases or disorders and ROS-mediated diseases or disorders. This invention also generally relates to compositions and to methods of using the compositions to increase tissue oxygenation.

In one aspect, the invention provides a composition comprising at least three of the group consisting of: a) hydroxytyrosol, b) resveratrol, c) lycopene, and d) flavanols or flavonoids.

Olives contain a number of bioactive compounds. Among these are tyrosol and hydroxytyrosol, hydrophilic phenolic alcohols. Hydroxytyrosol, believed to be the more important phenol, originates from the hydrolysis of a compound called oleuropein. Oleoropein develops during the maturation of olives. Small amounts of these compounds are found in virgin olive oils; their concentrations have been estimated to be 27.5 mg/kg and 14 mg/kg, respectively [Owen R et al. Identification of lignans as major components in the phenolic fraction of olive oil. Clin Chem 2000; 46: 976-988]. Spanish olive oils may contain as much as 381 mg/kg hydroxytyrosol [Brenes M et al Phenolic compounds in Spanish olive oils. J Agric Food Chem 1999; 47: 3535-40; Brenes M et al. Rapid and complete extraction of phenols from olive oil and determination by means of a colorimetric electrode array system. J Agric Food Chem. 2000; 48: 5178-83]. Phenolic alcohols are found in much greater quantities in aqueous waste streams from olive processing.

Population studies have shown improved cardiovascular health in persons consuming olive oil. For instance, dietary intake of olive oil phenolics by persons residing in Mediterranean countries is estimated to be approximately 9 mg per day with at least 1 mg from hydroxytyrosol and tyrosol [De la Torre R. Bioavailability of olive oil phenolic compounds in humans. Inflammopharmacology 2008; 16: 245-247]. Initial work assumed that the high monounsaturated fatty acid content of olive oils was responsible for this benefit. It is now understood that minor constituents (i.e. phenolic compounds) have biological activities that may contribute to heart health, which is captured in the Granados-Principal review of the history of research on olive oil and health [Granados-Principal S et al. Hydroxytyrosol: from laboratory investigations to future clinical trials. Nutr Rev 2010; 68: 191-206].

Hydroxytyrosol and tyrosol are absorbed in a dose-dependent manner with most absorption taking place in the small intestine and colon, while most elimination occurs in the urine as the corresponding glucuronide esters [Vissers M et al. Olive oil phenols are absorbed in humans. J Nutr 2002; 132: 409-417; Visioli F et al. Antioxidant properties of olive oil phenolics. In: Quiles J et al eds Olive Oil and Health. Oxford: CABI Publishing. 2006: 109-118]. Hydroxytyrosol is known to be absorbed very rapidly with plasma concentrations peaking 5-10 minutes after ingestion with relatively low bioavailability due to rapid elimination [Bai C et al. Determination of synthetic hydroxytyrosol in rat plasma by GC-MS. J Agric Food Chem. 1998 46: 3998-4001]. However, work by Visioli et al., 2003 suggests that uptake and elimination can be altered depending on the delivery vehicle (e.g., olive oil vs. yogurt altered the elimination profile) [Vision F, Riso P, Grande S, Galli C, Porrini M. Protective activity of tomato products on in vivo markers of lipid oxidation. Eur J Nutr 2003; 42: 201-206]. Following absorption, hydroxytyrosol associates with lipoproteins that deliver it to various tissues. Overall, there appear to be three metabolic fates for hydroxytyrosol: oxidation, methylation and methylation-oxidation [D'Angelo S et al. Pharmacokinetics and metabolism of hydroxytyrosol, a natural antioxidant from olive oil Drug Metab Dispos 2000; 29: 1492-98; Miro-Casas E et al. Hydroxytyrosol disposition in humans. Clin Chem 2003; 49: 945-952].

In vitro and animal studies have shown that hydroxytyrosol scavenges free radicals and chelate iron with a subsequent reduction in the generation or reactive oxygen species [Bovicelli P. Radical-scavenging polyphenols: new strategies for their synthesis. J Pharm Pharmacol 2007; 59: 1703-10]. Therefore, hydroxytyrosol may be useful in targeting diseases and disorders mediated by ROS (i.e., any of the diseases disclosed herein). For instance, such actions are important in preventing oxidation of lipoproteins which is a first step in producing atherosclerotic injury. Prevention of LDL oxidation has been confirmed in experimental models where phenolic compounds were introduced [Gonzalez-Santiago M et al. One month administration of hydroxytyrosol, a phenolic antioxidant present in olive oil, to hyperlipemic rabbits improves blood lipid profile, antioxidant status and reduces atherosclerosis development. Atherosclerosis 2006; 188: 35-42; Leenen R et al. supplementation of plasma with olive oil phenols and extracts: influence on LDL oxidation. J Agric Food Chem 2002; 50: 1290-97]. Similarly, rats fed hydroxytyrosol showed a reduction in plasma LDL and total cholesterol [Fki I et al. Hypocholesterolemic effects of phenolic extracts and purified hydroxytyrosol recovered from olive mill wastewater in rats fed a cholesterol-rich diet. J Agric Food Chem 2007; 55: 624-31; Jemai et al. Lipid-lowering and antioxidant effects of hydroxytyrosol and its triacetylated derivative recovered from olive tree leaves in cholesterol-fed rats. J Agric Food Chem 2008; 56: 2630-36].

Other potential cardio-protective actions shown in in vitro and animal work include a reduction in the expression of vascular adhesion molecules as well an effect on nitric oxide (NO) mediated vascular relaxation. In addition, hydroxytyrosol-treated rat myocytes showed an increase expression of sirtuins, which are known to be involved in moderating ROS [Mukherjee S et al. Expression of the longevity proteins by both red and white wines and their cardio-protective components, resveratrol, tyrosol and hydroxytyrosol. Free Rad Biol Med 2009; 46: 573-578].

In addition to its effect on ROS, hydroxytyrosol also appears to possess both anti-inflammatory and anti-thrombotic properties, which may contribute to cardio-protection and overall general health. Various in vitro models have establish that hydroxytyrosol reduces synthesis of Thromboxane B2, A2 and Leukotriene B4, all of which participate in regulating platelet aggregation and inflammation [Gonzalez-Correa J et al. Effects of hydroxytyrosol and hydroxytyrosol acetate administration to rats on platelet function compared to acetylsalicylic acid. J Agric Food Chem 2008; 56: 7872-76; Petroni A et al. Inhibition of platelet aggregation and eicosanoid production by phenolic components of olive oil. Thromb Res 1995; 78: 151-60]. In addition, hydroxytyrosol reduces platelet cAMP and CGMP platelet phosphodiesterase, regulators of platelet aggregation. Hydroxytyrosol appears to buffer free radical induced genotoxic damage in cell lines and red blood cells [Fabiani R et al. Oxidative DNA damage is prevented by extracts of olive oil, hydroxytyrosol and other olive phenolic compounds in human blood mononuclear cells and HL60 cells J Nutr 2008 138: 1411-16; Nousis L et al. DNA protecting and genotoxic effects of olive oil related components in cells exposed to hydrogen peroxide. Free Radic Res 2005; 39: 287-95; Palva-Martins F et al. Effects of olive oil polyphenols on erythrocyte oxidative damage. Mol Nutr Food Res. 2009 53: 609-16].

Cocoa is another important source of health promoting polyphenol compounds. The polyphenol type and content varies due to processing effects and growing conditions. In total, there are more than 380 compounds in cocoa. Most can be grouped into three broad classes of compounds: catechins, anthocyanidins and proanthocyanidins. Each class contributes to the bioactivity of the cocoa. Within each class, there are a number of compounds that vary in molecular size and structure. Even subtle differences may influence bioactivity and bioavailability. This relationship between structure and bioavailability has been reviewed recently in a paper by Andujar et al [Andujar M et al. Cocoa polyphenols and their potential benefits for human health. Oxidative Medicine and Cellular Longevity 2012; 906252].

A substantial number of studies have characterized the metabolic and cardiovascular effects of cocoa and its constituents (e.g., flavanols and flavonoids). For instance, cocoa polyphenols (e.g., flavanols) have antiplatelet effects, antioxidant and anti-inflammatory effects.

With regard to anti-inflammatory actions, epicatechin inhibits at least two steps in the conversion of arachidonic acid into leukotriene pro-inflammatory compounds, which have been confirmed in subjects consuming cocoa products [Sies H. et al. Cocoa polyphenols and inflammatory mediators. The American Journal of Clinical Nutrition 2005; 81: 304S-312S; Selmi C, et al. The anti-inflammatory properties of cocoa flavanols, Journal of Cardiovascular Pharmacology, 2006; 47: S163-S171). Selmi et al also summarized the experimental and clinical evidence that cocoa polyphenols have a targeted effect on reducing the production of proinflammatory IL-1B and IL-2 while promoting the production of anti-inflammatory cytokines IL-4 and TGF-B. Additional anti-inflammatory effects that have been characterized include Nuclear Factor activation and modulation of signal transcription. In experimental models, cocoa flavanols, such as epicatechin and catechin, inhibit NF-kB leading to a reduction in proinflammatory IL-2. Zhang et al (2006) observed that cocoa procyanidin dimers reduced expression of COX-2, an important enzyme in the inflammatory cascade [Zhang Y et al. Procyanidin dimer B2 [epicatechin-(4β-8)-epicatechin] suppresses the expression of cyclooxygenase-2 in endotoxin-treated monocytic cells. Biochemical and Biophysical Research Communications 2006; 345: 508-515].

Evidence in humans for a positive impact of cocoa polyphenols (e.g., flavanols) on cardiovascular disease is growing although it appears that the studies have not examined the role of cocoa compounds in reducing inflammation. Ding et al (2006) and Rimbach et al (2009) have reviewed some of the human cardiovascular clinical studies [Ding E et al. Chocolate and prevention of cardiovascular disease: a systematic review. Nutrition and Metabolism 2006; 3: article 2; Rimbach G et al. Polyphenols from cocoa and vascular health—a critical review. International Journal of Molecular Science. 2009; 10: 4290-4309]. Rimbach et al (2009) analyzed five specific epidemiologic trials and reported an inverse relationship between cardiovascular disease and cocoa intake. A meta-analysis by Cherniack (2011) concluded that cocoa consumption lowers blood pressure by 4.5 systolic and 2.5 diastolic mm Hg [Cherniack E. Polyphenols: planting the seeds of treatment for the metabolic syndrome. Nutrition 2011; 27: 617-623]. A meta-analysis by Desch et al (2010) also reported improved systolic and diastolic pressures in normotensive and pre-hypertensive subjects [Desch S et al. Effect of cocoa products on blood pressure: systematic review and meta-analysis. American Journal of Hypertension 2010; 23: 97-103]. Therefore, based on the foregoing, cocoa components (e.g., flavanols, flavonoids) are efficacious in positively impacting cardiovascular disease and, based on their anti-inflammatory properties, are proposed to be useful in treating, ameliorating, and/or preventing diseases or disorders containing an inflammation component (i.e., any of the diseases presented herein).

Resveratrol is a polyphenol found predominantly in grapes, peanuts, and Japanese knotweed, with the major dietary source of resveratrol being red wine. This compound initially came to light as a possible explanation for the French Paradox, where paradoxically the French people exhibit a relatively low incidence of cardiovascular disease, yet their diet comprises a large amount of saturated fats. Studies directed toward explaining this Paradox have identified a large number of biological actions that resveratrol has on cells. The resveratrol molecule can function as an antioxidant and it is known to express the synthesis of various antioxidant enzymes. In addition to its role in antioxidant systems, the molecule also appears to exert anti-inflammatory actions, which appear to be one of its main actions leading to improved health outcomes. As stated above, many researchers have already defined a link between inflammation and cardiovascular disease, obesity, metabolic syndrome, diabetes and other conditions. Therefore, resveratrol, which possesses anti-oxidant and anti-inflammatory activities, may be an important component in affecting diseases that are mediated by ROS or have an inflammatory component (i.e., any of the diseases presented herein).

With regard to its antioxidant properties, resveratrol from red wine or P. cuspidatum has been shown to decrease LDL oxidation, which may be important in limiting cardiovascular disease progression [Fuhrman, B., Lavy, A., Aviram, M., Consumption of red wine with meals reduces the susceptibility of human plasma and low-density lipoprotein to lipid peroxidation. Am J Clin Nutr 1995; 61: 549-554; Ghanim, H., Sia, C. L., Abuaysheh, S., Korzeniewski, K.et al. An anti-inflammatory and reactive oxygen species suppressive effects of an extract of Polygonum cuspidatum containing resveratrol. J. Clin. Endocrinol. Metab 2010; 95: E1-E8; Nigdikar, S. V. et al. Consumption of red wine polyphenols reduces the susceptibility of low-density lipoproteins to oxidation in vivo. Am J Clin Nutr 1998; 68: 258-265]. Ghanim et al (2010) found that treatment with resveratrol suppressed nuclear factor kappa B (NFkB) binding, decreased ROS generation, decreased tumor necrosis factor alpha (TNFa) and IL-6, two pro-inflammatory cytokines. Plasma TNFa and C-reactive protein (CRP) were also significantly reduced. These changes support the idea that resveratrol can reduce markers of inflammation and likely reduce cardiovascular disease risk.

In subjects consuming a high fat meal, resveratrol supplementation dampened the post-prandial rise in cell differentiation 14 (CD14) and IL-1b mRNA, and toll-like receptor 4 (TLR4) protein in mononuclear cells, while also decreasing plasma endotoxin, also strongly supporting the anti-inflammatory actions of resveratrol and their potential role in reducing cardiovascular disease. Petrowski (2011) reviewed other mechanisms by which resveratrol can offer cardiovascular health protection: 1) reduction in endothelin-1 and alteration in other adhesion molecules; 2) reduction in platelet aggregation and proinflammatory cytokines and an increase in NO synthesis; and 3) the activation of sirtuins [Petrowski G, Gurusamy N, Das D K. Resveratrol in cardiovascular health and disease. Ann NY Acad Sci 2011; 1215: 22-33].

Several recent large controlled clinical studies have confirmed the anti-inflammatory actions in populations with cardiovascular disease. In subjects with stable coronary artery disease, chronic daily consumption of a resveratrol-containing grape for one year increased serum adiponectin and prevented a PAI-1 increase, both favorable changes in atherothrombotic signaling [Tomé-Carneiro J et al. One-year supplementation with a grape extract containing resveratrol modulates inflammatory-related microRNAs and cytokines expression in peripheral blood mononuclear cells of type 2 diabetes and hypertensive patients with coronary artery disease. Pharmacol Res. 2013 Apr 1; 72C:69-82]. Another study found that 1-year supplementation with a resveratrol-rich grape supplement improved the inflammatory and fibrinolytic status (decrease in CRP, TNF-alpha, plasminogen activator inhibitor) in patients who were on statins for primary prevention of cardiovascular disease and/or at high risk (i.e., with diabetes or hypercholesterolemia plus ≧1 other risk factor) [Tomé-Carneiro J et al. One-year consumption of a grape nutraceutical containing resveratrol improves the inflammatory and fibrinolytic status of patients in primary prevention of cardiovascular disease. Am J Cardiol 2012; 110:356-63]. Additional clinical work by the same group, established that one year supplementation with grape resveratrol down regulated monocyte pro-inflammatory cytokines in a coronary artery disease population with hypertension and diabetes [Tomé-Carneiro J et al. One-year supplementation with a grape extract containing resveratrol modulates inflammatory-related microRNAs and cytokines expression in peripheral blood mononuclear cells of type 2 diabetes and hypertensive patients with coronary artery disease. Pharmacol Res. 2013 Apr 1; 72C:69-82].

Lycopene is a carotenoid responsible for the red color in tomatoes, watermelon, pink grapefruit, papaya, guava and rosehips, with the deepness of the red color generally correlating with the lycopene content in foods [Maiani G, Caston M J, Catasta G, et al. Carotenoids: actual knowledge on food sources, intakes, stability and bioavailability and their protective role in humans. Mol Nutr Food Res 2009; 53(Suppl 2):S194-218]. It has been shown that heating and homogenization are known to increase lycopene bioavailability from food sources [Porrini M, Riso P, Testolin G. Absorption of lycopene from single or daily portions of raw and processed tomato. Br J Nutr 1998; 218: 101-105; Shi J, Le Maguer M. Lycopene in tomatoes: chemical and physical properties affected by food processing. Crit Rev Biotechnol 2000; 20:293-334]. Research suggests that bioavailability is also enhanced in a lipid matrix and one study suggests oils containing monounsaturated fats may be advantageous as carriers [Basu A, Imrhan V. Tomatoes versus lycopene in oxidative stress and carcinogenesis: conclusions from clinical trials. Eur J Clin Nutr 2007; 61:295-303; Clarke R M, Yao I, She L, Furr H C. A comparison of lycopene and astaxanthin absorption from corn oil and olive oil emulsions. Lipids 2000; 35: 803-806; Lee A, Thurnham D I, Chopra M. Consumption of tomato products with olive oil but not sunflower oil increases the antioxidant activity of plasma. Free Radic Biol Med 2000; 29: 1051-1055; Gustin D M, Rodvold K A, Sosman J A et al. Single-dose pharmacokinetic study of lycopene delivered in a well-defined food-based lycopene delivery system (tomato paste-oil mixture) in healthy adult male subjects. Cancer Epidemiol Biomarkers Prey 2004; 13: 850-860].

Lycopene is the most abundant carotenoid consumed in the diet and is also the most abundant carotenoid found in blood. Intakes of lycopene in the US are wide ranging. Story et al (2010) reported an average intake of 5.7-10.5 mg/d for adult men and women. (As a reference, raw tomato provides 4.6 mg lycopene per cup) [Story E N, Kopec R E, Schwartz S J, Harris G K. An update on the health effects of tomato lycopene. Ann Rev Food Sci Technol. 2010; 1:189-210].

Lycopene's structure contains eleven conjugated double bonds which enable it to function as a potent antioxidant. In foods, the substance exists largely in the trans-form, while plasma lycopene contains a substantial quantity of cis-isomers. Lycopene has been shown in vitro to be an effective singlet oxygen quencher. In addition, it scavenges other reactive oxygen molecules such as superoxide, peroxyl and hydroxyl radicals. Numerous studies have confirmed in vivo antioxidant effects on LDL cholesterol oxidation and other markers, thus, suggesting that lycopene may be effective at treating not only cardiovascular disease (i.e., lower LDL cholesterol levels) but may also be an important component in a therapy targeting diseases or disorders mediated by ROS (i.e., any of the diseases or disorders presented herein).

Lycopene has the potential to improve cardiovascular health via several select actions: 1) reduction in serum lipids; 2) reduction in LDL oxidation and other oxidation markers; 3) alterations in platelet aggregation; and 4) modification of inflammation and immune responses. With respect to the mechanism or mechanisms that lycopene may employ in altering cholesterol metabolism, at least 3 mechanisms have been proposed: 1) suppression of cholesterol synthesis; 2) increased degradation of LDL; and 3) inhibition of HMG-CoA-reductase, a key enzyme in cholesterol synthesis.

### BRIEF DESCRIPTION OF THE INVENTION

In one aspect, the invention provides a composition comprising at least three of the group consisting of: a) hydroxytyrosol, b) resveratrol, c) lycopene, and d) flavanols or flavonoids. In certain aspects, the composition comprises hydroxytyrosol, resveratrol, and lycopene. In certain instances, the composition comprises about 6 mg hydroxytyrosol, about 25 mg resveratrol, and about 1.65 mg lycopene.

In other aspects, the invention provides a composition comprising hydroxytyrosol, resveratrol, lycopene, and flavanols. In a preferred embodiment, the composition comprises about 5-100 mg hydroxytyrosol (more preferably about 6 mg); about 25-500 mg resveratrol (more preferably about 25 mg); about 1-50 mg lycopene (more preferably about 1.65 mg); and about 25-2,000 mg flavanols (more preferably about 250 mg).

In any of the embodiments presented herein, hydroxytyrosol is isolated, extracted, or concentrated from olive water, olive pulp, olive oil, olive leaf, or a synthetic source (more preferably from olive water or olive pulp).

In any of the embodiments presented herein, resveratrol is isolated, extracted, or concentrated from Japanese Knotweed, red wine, red grape juice, grapes, peanuts, cocoa, chocolate, or a synthetic source.

In any of the embodiments presented herein, lycopene is isolated, extracted, or concentrated from tomatoes, microbial production via fermentation, guava, grapefruit, parsley, basil, persimmons (more preferably from tomatoes), or a synthetic source.

In any of the embodiments presented herein, flavanols are isolated, extracted, or concentrated from cocoa, chocolate, or tea (more preferably from cocoa or chocolate).

In any of the embodiments presented herein, flavonoids are isolated, extracted, or concentrated from acacia tree heartwood (acacia catechu), berries (e.g., blueberries, cranberries, or cherries), tree fruits (e.g., bananas or citrus fruits), beans (e.g., black beans or kidney beans), tree nuts (e.g., cashews, walnuts, or pecans), green vegetables (e.g., broccoli or green peppers), red vegetables (e.g., red peppers), or a synthetic source.

In other aspects, the invention provides a composition comprising hydroxytyrosol, resveratrol, lycopene, and flavonoids. In a preferred embodiment, the composition comprises about 5-100 mg hydroxytyrosol (more preferably about 6 mg); about 25-500 mg resveratrol (more preferably about 25 mg); about 1-50 mg lycopene (more preferably about 1.65 mg); and about 25-2,000 mg flavonoids (more preferably about 250 mg).

In any of the embodiments presented herein, any of the compositions present herein may further comprise one or more of the group consisting of rosemary extract, oregano extract, apple cider vinegar powder, grape seed extract, broccoli juice concentrate, carrot juice concentrate, tomato juice concentrate, beet juice concentrate, spinach juice concentrate, cucumber juice concentrate, brussel sprout juice concentrate, cabbage juice concentrate, celery juice concentrate, kale juice concentrate, asparagus juice concentrate, green bell pepper juice concentrate, cauliflower juice concentrate, parsley juice concentrate, and wheat grass juice concentrate.

In another aspect, this invention relates to a multicomponent formula, where each component has anti-inflammatory activity and/or antioxidant activity. The components each have a specific mode of action in the body, and their anti-inflammatory and/or antioxidant effects are complimentary. The components each have their own characteristic bioavailability and metabolism. It is important to note that the compositions covered in this invention optimize effectiveness by combining components with complementary actions. Components of the formulas also have different pharmacokinetic properties. Combining select components enhances their overall effectiveness. Combining these components also allows one product to offer a broad approach to managing the complex inflammatory and/or oxidative stress responses.

In another aspect, the invention provides a method of improving at least one risk factor in a subject possessing said risk factor or risk factors comprising administering to said subject an effective amount of any composition presented herein, wherein:

the risk factors are selected from the group consisting of elevated C-reactive protein, hypertension, elevated low-density lipoprotein (LDL) cholesterol levels, low high-density lipoprotein (HDL) cholesterol levels, elevated triglycerides, elevated tumor necrosis factor (TNF), low tissue oxygenation, elevated fasting or post-prandial insulin levels, elevated fasting or post-prandial glucose levels, elevated fasting or post-prandial hemoglobin A1c (HbA1c), elevated non-esterified free fatty acids (NEFAs), elevated body mass index (BMI), hypothyroidism, hyperthyroidism, and impaired cognition;

such that at least one of said risk factors is improved.

In any of the embodiments presented herein, the subject referred to therein may or may not suffer from one or more of diseases or disorders selected from the group consisting of cardiovascular disease, athlerosclerosis, heart failure, hypercholesterolemia, diabetes, inflammation associated with pain, and metabolic syndrome.

In another aspect, the invention provides a method of treating dyslipidemia in a subject comprising the administration to said subject an effective amount of any composition presented herein, such that said dyslipidemia is improved. In certain instances, the treatment affects one or more of the following parameters selected from the group consisting of: reducing total cholesterol levels, reducing LDL cholesterol levels, reducing levels of reactive oxygen species (ROS), reducing triglycerides, and increasing HDL cholesterol levels.

In another aspect, the invention provides a method of reducing inflammation in a subject comprising the administration to said subject an effective amount of any composition presented herein, such that said inflammation is improved. In certain instances, said subject is identified as having one or more parameters selected from the group consisting of: elevated C-reactive protein, elevated TNF, elevated LDL cholesterol levels, low HDL cholesterol levels, elevated triglycerides, low tissue oxygenation, elevated fasting or post-prandial insulin levels, elevated fasting or post-prandial glucose levels, elevated fasting or post-prandial HbA1c, elevated non-esterified free fatty acids (NEFAs), and elevated body mass index (BMI). In certain instances, the inflammation (e.g., joint inflammation, reduced range of motion, or joint pain) or inflammation-mediated disease or disorder (e.g., osteoarthritis or rheumatoid arthritis) is associated with pain.

In certain instances, the inflammation or inflammation-mediated disease or disorder delineated in any embodiment presented herein is skin inflammation (e.g., eczema or psoriasis). In a further embodiment, the treatment of said inflammation or inflammation-mediated disease or disorder affects at least one of the parameters selected from the group consisting of: reducing transcutaneous pressure of oxygen, increasing oxygen saturation in the microcirculation (StO2), reducing skin redness or skin irritation, and reducing ROS.

In any of the embodiments presented herein, the treatment of any of the diseases or disorders presented herein with an effective amount of any of the compositions presented herein results in lowering of ROS.

In another aspect, the invention provides a method of treating hyperglycemia in a subject identified as suffering from hyperglycemia comprising the administration to said subject an effective amount of any composition presented herein, such that said hyperglycemia is improved. In certain instances the hyperglycemia is identified in the subject using fasted or post-prandial glucose measurements.

In another aspect, the invention provides a method of treating hyperinsulinemia in a subject identified as suffering from hyperinsulinemia comprising the administration to said subject an effective amount of any composition presented herein, such that said hyperinsulinemia is improved. In certain instances the hyperinsulinemia is identified in the subject using fasted or post-prandial insulin measurements.

In another aspect, the invention provides a method of reducing elevated HbA1c levels in a subject identified as suffering from elevated HbA1c levels comprising the administration to said subject an effective amount of any composition presented herein, such that said elevated HbA1c levels are reduced. In certain instances the elevated HbA1c levels are identified in the subject using fasted or post-prandial HbA1c measurements.

In another aspect, the invention provides a method of treating diabetes in a subject identified as suffering from diabetes comprising the administration to said subject an effective amount of any composition presented herein, such that said diabetes is improved. In certain instances the treatment reduces one or more parameters selected from the group consisting of glucose levels, insulin levels, triglycerides, NEFAs, ROS levels, and HbA1c.

In another aspect, the invention provides a method of treating cardiovascular disease in a subject identified as suffering from cardiovascular disease comprising the administration to said subject an effective amount of any composition presented herein, such that said cardiovascular disease is improved. In certain instances the treatment reduces one or more parameters selected from the group consisting of blood pressure, athlerosclerosis, platelet aggregation, total cholesterol levels, ROS levels, C-reactive protein, TNF, BMI, triglycerides, and LDL cholesterol levels.

In another aspect, the invention provides a method of reducing ROS in a subject comprising the administration to said subject an effective amount of any composition presented herein.

In another aspect, the invention provides a method of reducing platelet aggregation in a subject comprising the administration to said subject an effective amount of any composition presented herein.

In any of the embodiments presented herein, ROS is oxygen radical, superoxide, or singlet oxygen.

In another aspect, the invention provides a method of increasing total antioxidant capacity in a subject comprising the administration to said subject an effective amount of any composition presented herein.

In another aspect, the invention provides a method of treating aging or a disease or disorder associated with aging (e.g., arthritis, osteoarthritis, rheumatoid arthritis, cancer, proliferative diseases, dementia, Alzheimer's disease, Parkinson's disease, hypertension, hypercholesterolemia, diabetes, cardiovascular disease, stroke, and heart disease) in a subject comprising the administration to said subject an effective amount of any composition presented herein. In certain instances, the aging or the disease or disorder associated with aging is mediated through one or more of sirtuin pathways (e.g., Sirt1 or Sirt3).

In another aspect, the invention provides a method of treating a cognitive disorder (e.g., impaired memory, dementia, or Alzheimer's disease) in a subject comprising the administration to said subject an effective amount of any composition presented herein, such that said cognitive disorder is improved.

In another aspect, the invention provides a method of treating a subject at risk of developing diabetes, said method comprising:

a. identifying said subject as at risk of developing diabetes (e.g., one or more of parameters selected from the group consisting of elevated glucose levels, elevated insulin levels, elevated HbA1c levels, elevated NEFAs, elevated triglycerides, and elevated BMI); and

b. administering to said subject an effective amount of any composition presented herein.

In another aspect, a subject suffers from diabetes wherein at least one of the following conditions is fulfilled: a) HbA1c measurement is at least 6.5%; b) fasting glucose levels are at least 126 mg/dL; or c) post-prandial (i.e., oral glucose tolerance test) glucose levels are at least 200 mg/dL.

In another aspect, a subject is identified as suffering from pre-diabetes wherein at least one of the following conditions is fulfilled: a) HbA1c measurement is ranges from about 5.7% to 6.4%; b) fasting glucose levels ranges from about 100-125 mg/dL; or c) post-prandial (i.e., oral glucose tolerance test) glucose levels ranges from about 140-199 mg/dL.

In another aspect, the invention provides a method of treating a subject at risk of developing cardiovascular disease, said method comprising:

a. identifying said subject as at risk of developing cardiovascular disease (e.g., one or more of the parameters selected from the group consisting of hypertension, elevated total cholesterol levels, elevated LDL cholesterol levels, low HDL cholesterol levels, elevated C-reactive protein levels, elevated TNF levels, elevated triglycerides, elevated BMI, and atherosclerosis); and

b. administering to said subject an effective amount of any composition presented herein.

In another aspect, a subject suffers from elevated total cholesterol when said subject's total cholesterol measurement is at least 240 mg/dL.

In another aspect, a subject suffers from elevated LDL cholesterol when said subject's LDL cholesterol measurement is at least 160 mg/dL.

In another aspect, a subject suffers from low HDL cholesterol when said subject's HDL cholesterol measurement is less than 40 mg/dL for males and 50 mg/dL for females.

In another aspect, a subject suffers from elevated triglycerides when said subject's tryglyceride measurement is at least 200 mg/dL.

In another aspect, the invention provides a method of treating obesity in a subject comprising the administration to said subject an effective amount of any composition presented herein, such that said obesity is improved. In certain instances, said treatment affects one or more of the following parameters selected from the group consisting of: reducing body weight, reducing percent body fat, reducing BMI, reducing fasting glucose levels, reducing post-prandial glucose levels, reducing fasting insulin levels, reducing post-prandial insulin levels, and reducing ROS levels.

In another aspect, the invention provides a method of treating or preventing skin inflammation due to sun exposure in a subject comprising the administration to said subject an effective amount of any composition presented herein. In certain instances, said treatment reduces a) recovery time from sunburn or b) ROS.

In another aspect, the invention provides a method of treating an allergic reaction (e.g., due to a food allergy) in a subject comprising the administration to said subject an effective amount of any composition presented herein, such that said allergic reaction is reduced. In certain instances, the treatment reduces at least of one of the following parameters selected from the group consisting of: a) blood antibodies in response to said allergy, b) eosinophil levels, and c) ROS.

In another aspect, the invention provides a method of enhancing tissue (e.g., skin or muscle) oxygenation in a subject comprising the administration to said subject an effective amount of any composition presented herein. In certain instances, the treatment affects at least one of the parameters selected from the group consisting of: a) increasing blood hemoglobin oxygen saturation (e.g., SO2, SaO2), b) decreasing partial pressure of oxygen (SpO2), c) decreasing transcutaneous pressure of oxygen, d) improvement in VO2 measurement, and e) increasing oxygen saturation in the microcirculation (StO2). In other aspects, the enhanced tissue oxygenation improves fitness level or exercise performance in said subject.

In another aspect, the invention provides a method of treating hyperthyroidism in a subject comprising the administration to said subject an effective amount of any composition presented herein, such that said hyperthyroidism is improved.

In another aspect, the invention provides a method of treating hypothyroidism in a subject comprising the administration to said subject an effective amount of any composition presented herein, such that said hypothyroidism is improved.

In another aspect, the invention provides a method of enhancing athletic performance in a subject comprising the administration to said subject an effective amount of any composition presented herein. In other aspects, the method improves walking speed, running speed, strength, endurance, or VO2 measurement

In certain embodiments, the methods are useful in providing and/or enhancing anti-aging properties of skin by preventing (e.g., UVA-induced, UVB-induced, photo-damage, aging) wrinkle formation. In certain embodiments, the methods herein are useful in providing and/or enhancing skin tone and skin appearance properties of skin by administration of compositions delineated herein.

In certain embodiments, the compounds and compositions herein are useful in providing and/or enhancing anti-aging properties of skin by preventing (e.g., UVA-induced, UVB-induced, photo-damage, aging) wrinkle formation. In certain embodiments, the compounds and compositions herein are useful in providing and/or enhancing skin tone and skin appearance properties of skin by administration of any composition delineated herein.

In certain embodiments, the subject is a mammal, preferably a primate or human.

In another embodiment, the invention provides a method as described above, wherein the effective amount of the compound or composition (e.g., any composition delineated herein) ranges from about 0.005 μg/kg to about 500 mg/kg, preferably about 0.1 mg/kg to about 500 mg/kg, more preferably about 10 mg/kg to about 500 mg/kg of body weight.

In other embodiments, the invention provides a method as described above wherein the effective amount of the compound or composition (e.g., any composition delineated herein) ranges from about 1.0 nM to about 500 μM. In another embodiment, the effective amount ranges from about 100 nM to about 100 μM.

In other embodiments, the invention provides a method as described above wherein the effective amount of the compound or composition (e.g., any composition delineated herein) ranges from about 0.1 mg/ml to about 1000 mg/ml. In certain embodiments, the effective amount ranges from about 1.0 mg/ml to about 500 mg/ml. In another embodiment, the effective amount ranges from about 1.0 mg/ml to about 100 mg/ml.

In another embodiment, the invention provides a method as described above, wherein the compound or composition (e.g., any composition delineated herein) is administered intravenously, intramuscularly, subcutaneously, intracerebroventricularly, orally or topically.

Another object of the present invention is the use of a compound or composition (e.g., any composition delineated herein) as described herein for use in the treatment and/or prevention of an inflammatory- or ROS-mediated disorder or disease. Another object of the present invention is where the disease or disorder includes proliferative diseases and disorders, inflammation, cancer, Alzheimer's disease and other neurodegenerative disorders, stroke, chronic kidney disease, type II diabetes, and aging itself, and other diseases mediated through ROS or inflammation.

In one aspect, the invention provides a kit comprising an effective amount of a compound or composition (e.g., any composition delineated herein), in unit dosage form, together with instructions for administering the compound to a subject suffering from or susceptible to an inflammatory- or ROS-mediated disease or disorder, including proliferative diseases and disorders, inflammation, cancer, stroke, chronic kidney disease, type II diabetes, and aging itself, and other diseases mediated through ROS or inflammation, Alzheimer's disease and other neurodegenerative disorders, memory loss, inducing neurogenesis, enhancing memory retention, enhancing memory formation, increasing synaptic potential or transmission, or increasing long term potentiation (LTP), etc.

Methods delineated herein include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

### DETAILED DESCRIPTION

### Definitions

In order that the invention may be more readily understood, certain terms are first defined here for convenience.

As used herein, the term “at risk” of developing a certain disease or disorder (e.g., diabetes or cardiovascular disease) encompasses possessing any risk factor, susceptibility, or predisposition of developing a certain disease.

As used herein, the term “treating” a disorder encompasses preventing, ameliorating, mitigating and/or managing the disorder and/or conditions that may cause the disorder. The terms “treating” and “treatment” refer to a method of alleviating or abating a disease and/or its attendant symptoms. In accordance with the present invention “treating” includes preventing, blocking, inhibiting, attenuating, protecting against, modulating, reversing the effects of and reducing the occurrence of e.g., the harmful effects of a disorder.

As used herein, “inhibiting” encompasses preventing, reducing and halting progression.

As used herein, “activating” encompasses permitting, increasing and enhancing progression.

As used herein, “enriched” encompasses greater or increased amounts of a material or desired or active compound or agent relative to its natural or other reference state.

As used herein, as “extract” is a preparation of constituents of a material (e.g., seaweed), including for example, solvent extracts, concentrated forms of said constituents, concentrated solvent extracts, isolated chemical compounds or mixtures thereof.

The term “modulate” refers to increases or decreases in the activity of a cell in response to exposure to a compound of the invention.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

A “peptide” is a sequence of at least two amino acids. Peptides can consist of short as well as long amino acid sequences, including proteins.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The term “protein” refers to series of amino acid residues connected one to the other by peptide bonds between the alpha-amino and carboxy groups of adjacent residues.

Amino acids may be referred to herein by either their commonly known three letter symbols or by the one-letter symbols recommended by the IUPAC-IUB Biochemical Nomenclature Commission.

As to amino acid sequences, one of skill will recognize that individual substitutions, deletions or additions to a peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

Macromolecular structures such as polypeptide structures can be described in terms of various levels of organization. For a general discussion of this organization, see, e.g., Alberts et al., Molecular Biology of the Cell (3rd ed., 1994) and Cantor and Schimmel, Biophysical Chemistry Part I. The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that form a compact unit of the polypeptide and are typically 50 to 350 amino acids long. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

The term “administration” or “administering” includes routes of introducing the compound(s) to a subject to perform their intended function. Examples of routes of administration which can be used include injection (subcutaneous, intravenous, parenterally, intraperitoneally, intrathecal), topical, oral, inhalation, rectal and transdermal.

The term “effective amount” includes an amount effective, at dosages and for periods of time necessary, to achieve the desired result. An effective amount of compound may vary according to factors such as the disease state, age, and weight of the subject, and the ability of the compound to elicit a desired response in the subject. Dosage regimens may be adjusted to provide the optimum therapeutic response. An effective amount is also one in which any toxic or detrimental effects (e.g., side effects) of compositions presented herein are outweighed by the therapeutically beneficial effects.

The phrases “systemic administration,” “administered systemically”, “peripheral administration” and “administered peripherally” as used herein mean the administration of a compound(s), drug or other material, such that it enters the patient's system and, thus, is subject to metabolism and other like processes.

The term “therapeutically effective amount” refers to that amount of the compound being administered sufficient to prevent development of or alleviate to some extent one or more of the symptoms of the condition or disorder being treated.

A therapeutically effective amount of compound (i.e., an effective dosage) may range from about 0.005 μg/kg to about 1000 mg/kg, preferably about 0.1 mg/kg to about 1000 mg/kg, more preferably about 10 mg/kg to about 500 mg/kg of body weight. In other embodiments, the therapeutically effective amount may range from about 0.10 nM to about 500 μM. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a compound can include a single treatment or, preferably, can include a series of treatments. It will also be appreciated that the effective dosage of a compound used for treatment may increase or decrease over the course of a particular treatment.

The term “diastereomers” refers to stereoisomers with two or more centers of dissymmetry and whose molecules are not minor images of one another.

The term “enantiomers” refers to two stereoisomers of a compound which are non-superimposable minor images of one another. An equimolar mixture of two enantiomers is called a “racemic mixture” or a “racemate.”

The term “isomers” or “stereoisomers” refers to compounds which have identical chemical constitution, but differ with regard to the arrangement of the atoms or groups in space.

The term “prodrug” includes compounds with moieties which can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionoic acid esters and acyl esters. Prodrugs which are converted to active forms through other mechanisms in vivo are also included. In aspects, the compounds of the invention are prodrugs of any of the formulae herein.

The term “subject” refers to animals such as mammals, including, but not limited to, primates (e.g., humans), cows, sheep, goats, horses, dogs, cats, rabbits, rats, mice and the like. In certain embodiments, the subject is a human.

Furthermore the compounds of the invention include olefins having either geometry: “Z” refers to what is referred to as a “cis” (same side) conformation whereas “E” refers to what is referred to as a “trans” (opposite side) conformation. With respect to the nomenclature of a chiral center, the terms “d” and “l” configuration are as defined by the IUPAC Recommendations. As to the use of the terms, diastereomer, racemate, epimer and enantiomer, these will be used in their normal context to describe the stereochemistry of preparations.

### Compounds of the Invention

Compounds (e.g., isolated compounds, compounds within extracts, compounds fractionated from extracts) of the invention can be made by means known in the art of organic synthesis. Methods for optimizing reaction conditions, if necessary minimizing competing by-products, are known in the art. Reaction optimization and scale-up may advantageously utilize high-speed parallel synthesis equipment and computer-controlled microreactors (e.g. Design And Optimization in Organic Synthesis, 2^{nd} Edition, Carlson R, Ed, 2005; Elsevier Science Ltd.; Jáhnisch, K et al, Angew. Chem. Int. Ed. Engl. 2004 43: 406; and references therein). Additional reaction schemes and protocols may be determined by the skilled artesian by use of commercially available structure-searchable database software, for instance, SciFinder® (CAS division of the American Chemical Society) and CrossFire Beilstein® (Elsevier MDL), or by appropriate keyword searching using an internet search engine such as Google® or keyword databases such as the US Patent and Trademark Office text database.

The compounds herein may also contain linkages (e.g., carbon-carbon bonds) wherein bond rotation is restricted about that particular linkage, e.g. restriction resulting from the presence of a ring or double bond. Accordingly, all cis/trans and E/Z isomers are expressly included in the present invention. The compounds herein may also be represented in multiple tautomeric forms, in such instances, the invention expressly includes all tautomeric forms of the compounds described herein, even though only a single tautomeric form may be represented. All such isomeric forms of such compounds herein are expressly included in the present invention. All crystal forms and polymorphs of the compounds described herein are expressly included in the present invention. Also embodied are extracts and fractions comprising compounds of the invention. The term isomers is intended to include diastereoisomers, enantiomers, regioisomers, structural isomers, rotational isomers, tautomers, and the like. For compounds which contain one or more stereogenic centers, e.g., chiral compounds, the methods of the invention may be carried out with an enantiomerically enriched compound, a racemate, or a mixture of diastereomers.

The present invention also contemplates solvates (e.g., hydrates) of a compound of herein, compositions thereof, and their use in the treatment and/or prevention of inflammatory- or reactive oxygen species (ROS)-mediated diseases. As used herein, “solvate” refers to the physical association of a compound of the invention with one or more solvent or water molecules, whether organic or inorganic. In certain instances, the solvate is capable of isolation, for example, when one or more solvate molecules are incorporated in the crystal lattice of the crystalline solid.

Preferred enantiomerically enriched compounds have an enantiomeric excess of 50% or more, more preferably the compound has an enantiomeric excess of 60%, 70%, 80%, 90%, 95%, 98%, or 99% or more. In preferred embodiments, only one enantiomer or diastereomer of a chiral compound of the invention is administered to cells or a subject.

### Pharmaceutical Compositions

The term “pharmaceutically acceptable salts” or “pharmaceutically acceptable carrier” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts. Other pharmaceutically acceptable carriers known to those of skill in the art are suitable for the present invention.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

The invention also provides a pharmaceutical composition, comprising an effective amount a compound described herein and a pharmaceutically acceptable carrier. In an embodiment, compound is administered to the subject using a pharmaceutically-acceptable formulation, e.g., a pharmaceutically-acceptable formulation that provides sustained delivery of the compound to a subject for at least 12 hours, 24 hours, 36 hours, 48 hours, one week, two weeks, three weeks, or four weeks after the pharmaceutically-acceptable formulation is administered to the subject.

Actual dosage levels and time course of administration of the active ingredients in the pharmaceutical compositions of this invention may be varied so as to obtain an amount of the active ingredient which is effective to achieve the desired therapeutic response for a particular patient, composition, and mode of administration, without being toxic (or unacceptably toxic) to the patient.

In use, at least one compound according to the present invention is administered in a pharmaceutically effective amount to a subject in need thereof in a pharmaceutical carrier by intravenous, intramuscular, subcutaneous, or intracerebro ventricular injection or by oral administration or topical application. In accordance with the present invention, a compound of the invention may be administered alone or in conjunction with a second, different therapeutic. By “in conjunction with” is meant together, substantially simultaneously or sequentially. In one embodiment, a compound of the invention is administered acutely. The compound of the invention may therefore be administered for a short course of treatment, such as for about 1 day to about 1 week. In another embodiment, the compound of the invention may be administered over a longer period of time to ameliorate chronic disorders, such as, for example, for about one week to several months depending upon the condition to be treated.

By “pharmaceutically effective amount” as used herein is meant an amount of a compound of the invention, high enough to significantly positively modify the condition to be treated but low enough to avoid serious side effects (at a reasonable benefit/risk ratio), within the scope of sound medical judgment. A pharmaceutically effective amount of a compound of the invention will vary with the particular goal to be achieved, the age and physical condition of the patient being treated, the severity of the underlying disease, the duration of treatment, the nature of concurrent therapy and the specific organozinc compound employed. For example, a therapeutically effective amount of a compound of the invention administered to a child or a neonate will be reduced proportionately in accordance with sound medical judgment. The effective amount of a compound of the invention will thus be the minimum amount which will provide the desired effect.

A decided practical advantage of the present invention is that the compound may be administered in a convenient manner such as by intravenous, intramuscular, subcutaneous, oral or intra-cerebroventricular injection routes or by topical application, such as in creams or gels. Depending on the route of administration, the active ingredients which comprise a compound of the invention may be required to be coated in a material to protect the compound from the action of enzymes, acids and other natural conditions which may inactivate the compound. In order to administer a compound of the invention by other than parenteral administration, the compound can be coated by, or administered with, a material to prevent inactivation.

The compound may be administered parenterally or intraperitoneally. Dispersions can also be prepared, for example, in glycerol, liquid polyethylene glycols, and mixtures thereof, and in oils.

The pharmaceutical forms suitable for injectable use include sterile aqueous solutions (where water soluble) or dispersions and sterile powders for the extemporaneous preparation of sterile injectable solutions or dispersions. In all cases the form must be sterile and must be fluid to the extent that easy syringability exists. It must be stable under the conditions of manufacture and storage. The carrier can be a solvent or dispersion medium containing, for example, water, DMSO, ethanol, polyol (for example, glycerol, propylene glycol, liquid polyethylene glycol, and the like), suitable mixtures thereof and vegetable oils. The proper fluidity can be maintained, for example, by the use of a coating such as lecithin, by the maintenance of the required particle size in the case of dispersion. In many cases it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the compound of the invention in the required amount in the appropriate solvent with various of the other ingredients enumerated above, as required, followed by filtered sterilization. Generally, dispersions are prepared by incorporating the various sterilized compounds into a sterile vehicle which contains the basic dispersion medium and the required other ingredients from those enumerated above. In the case of sterile powders for the preparation of sterile injectable solutions, the preferred methods of preparation are vacuum-drying and the freeze-drying technique which yields a powder of the active ingredient plus any additional desired ingredient from previously sterile-filtered solution thereof.

For oral therapeutic administration, the compound may be incorporated with excipients and used in the form of ingestible tablets, buccal tablets, troches, capsules, elixirs, suspensions, syrups, wafers, and the like. Compositions or preparations according to the present invention are prepared so that an oral dosage unit form contains compound concentration sufficient to treat a disorder in a subject.

Some examples of substances which can serve as pharmaceutical carriers are sugars, such as lactose, glucose and sucrose; starches such as corn starch and potato starch; cellulose and its derivatives such as sodium carboxymethycellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil, corn oil and oil of theobroma; polyols such as propylene glycol, glycerine, sorbitol, manitol, and polyethylene glycol; agar; alginic acids; pyrogen-free water; isotonic saline; and phosphate buffer solution; skim milk powder; as well as other non-toxic compatible substances used in pharmaceutical formulations such as Vitamin C, estrogen and echinacea, for example. Wetting agents and lubricants such as sodium lauryl sulfate, as well as coloring agents, flavoring agents, lubricants, excipients, tableting agents, stabilizers, anti-oxidants and preservatives, can also be present.

Topical administration of the pharmaceutical compositions of this invention is especially useful when the desired treatment involves areas or organs readily accessible by topical application. For topical application topically to the skin, the pharmaceutical composition should be formulated with a suitable ointment, lotion, or cream containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax, and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol, and water. The pharmaceutical compositions of this invention may also be topically applied to the lower intestinal tract by rectal suppository formulation or in a suitable enema formulation. Topically-transdermal patches and iontophoretic administration are also included in this invention.

For topical administration, the active compound(s), extracts, enriched extracts, or prodrug(s) can be formulated as solutions, gels, ointments, creams, suspensions, and the like.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

### EXAMPLES

The present invention will now be demonstrated using specific examples that are not to be construed as limiting.

### Example 1

### Composition Comprising Hydroxytyrosol, Resveratrol, and Lycopene Reduces Total Cholesterol

A subject was administered a composition comprising 6 mg hydroxytyrosol, 25 mg resveratrol, and 1.65 mg lycopene along with a 297 mg blend of Rosemary (Rosmarinus Officinalis) Extract, Oregano (Origanum Vulgare) Extract, Apple Cider Vinegar Powder, Grape Seed Extract, Broccoli Juice Concentrate, Carrot Juice Concentrate, Tomato Juice Concentrate, Beet Juice Concentrate, Spinach Juice Concentrate, Cucumber Juice Concentrate, Brussel Sprout Juice Concentrate, Cabbage Juice Concentrate, Celery Juice Concentrate, Kale Juice Concentrate, Asparagus Juice Concentrate, Green Bell Pepper Juice Concentrate, Cauliflower Juice Concentrate, Parsley Juice Concentrate, and Wheat Grass Juice Concentrate twice/day for 42 days. After 3 weeks of the dosing regimen (i.e., 21 days of bid dosing), the subject's HDL cholesterol was raised from 55 mg/dL to 65 mg/dL. After an additional contiguous 3 weeks of the dosing regimen (i.e., total of 42 days of bid dosing), the subject's HDL cholesterol was raised further from 65 mg/dL to 71 mg/dL. During the total 42 days of bid dosing the subject's total cholesterol was reduced by 60 points from 280 mg/dL to 220 mg/dL. The starting total cholesterol level was measured on day 1 (i.e., the day before dosing began). Another subject was administered the same composition at the same dosing and after 51 days of continuous use had total cholesterol reduced from 204 mg/dL to 150 mg/dL and LDL cholesterol reduce from 136 mg/dL to 80 mg/dL.

### Example 2

### Composition Comprising Hydroxytyrosol, Resveratrol, and Lycopene Restores Thyroid Function

A subject diagnosed with a damaged hypothalamus, which had been unsuccessfully treated (resulting in lack of thyroid function) for greater than 15 years, was administered a composition comprising 6 mg hydroxytyrosol, 25 mg resveratrol, and 1.65 mg lycopene along with a 297 mg blend of Rosemary (Rosmarinus Officinalis) Extract, Oregano (Origanum Vulgare) Extract, Apple Cider Vinegar Powder, Grape Seed Extract, Broccoli Juice Concentrate, Carrot Juice Concentrate, Tomato Juice Concentrate, Beet Juice Concentrate, Spinach Juice Concentrate, Cucumber Juice Concentrate, Brussel Sprout Juice Concentrate, Cabbage Juice Concentrate, Celery Juice Concentrate, Kale Juice Concentrate, Asparagus Juice Concentrate, Green Bell Pepper Juice Concentrate, Cauliflower Juice Concentrate, Parsley Juice Concentrate, and Wheat Grass Juice Concentrate twice/day for 90 days. After 45 days of the dosing regimen (i.e. 45 days of bid dosing), the subject's reading of thyroid stimulating hormone (TSH) was 0.1 mIU/L. At the end of the dosing regimen (i.e., 90 days of bid dosing), the subject's reading of thyroid stimulating hormone (TSH) was 0.42 mIU/L, into the normal range. These data demonstrate that the composition is capable of restoring thyroid function, even where traditional medical intervention has failed.

### Example 3

### Composition Comprising Hydroxytyrosol, Resveratrol, and Lycopene Reduces Joint Pain and Increases Mobility

A subject suffering from joint pain and inflammation which prevented him from swinging a golf club at all was administered a composition comprising 6 mg hydroxytyrosol, 25 mg resveratrol, and 1.65 mg lycopene along with a 297 mg blend of Rosemary (Rosmarinus Officinalis) Extract, Oregano (Origanum Vulgare) Extract, Apple Cider Vinegar Powder, Grape Seed Extract, Broccoli Juice Concentrate, Carrot Juice Concentrate, Tomato Juice Concentrate, Beet Juice Concentrate, Spinach Juice Concentrate, Cucumber Juice Concentrate, Brussel Sprout Juice Concentrate, Cabbage Juice Concentrate, Celery Juice Concentrate, Kale Juice Concentrate, Asparagus Juice Concentrate, Green Bell Pepper Juice Concentrate, Cauliflower Juice Concentrate, Parsley Juice Concentrate, and Wheat Grass Juice Concentrate four times/day for 60 days. At the end of the dosing regimen (i.e., 60 days of bid dosing), the subject experienced substantial pain relief and enough restoration of motion to resume playing full rounds of golf without joint pain.

### Example 4

### Composition Comprising Hydroxytyrosol, Resveratrol, and Lycopene Prevents Allergic Reactions

Two subjects diagnosed with a severe tree nut allergy were administered a composition comprising 6 mg hydroxytyrosol, 25 mg resveratrol, and 1.65 mg lycopene along with a 297 mg blend of Rosemary (Rosmarinus Officinalis) Extract, Oregano (Origanum Vulgare) Extract, Apple Cider Vinegar Powder, Grape Seed Extract, Broccoli Juice Concentrate, Carrot Juice Concentrate, Tomato Juice Concentrate, Beet Juice Concentrate, Spinach Juice Concentrate, Cucumber Juice Concentrate, Brussel Sprout Juice Concentrate, Cabbage Juice Concentrate, Celery Juice Concentrate, Kale Juice Concentrate, Asparagus Juice Concentrate, Green Bell Pepper Juice Concentrate, Cauliflower Juice Concentrate, Parsley Juice Concentrate, and Wheat Grass Juice Concentrate four times a day for 90 days. One of the subjects had been allergic to nuts since childhood, while the other had more recently acquired nut allergies as an adult. Prior to the dosing regimen, skin prick tests and blood antibody tests had rated “5” for both subjects for nut allergy—the highest rating on a 5-point scale. At the end of the dosing regimen (i.e., 90 days of bid dosing), both subjects registered no allergy in skin prick tests or blood antibody tests for nuts and were able and continue to be able to ingest tree nuts, peanuts and peanut products safely without any allergic reaction or requiring any additional treatment.

All patents, patent applications, and published references cited herein are hereby incorporated by reference in their entirety.

While this invention has been particularly illustrated and described with reference to particular examples, it will be understood by those skilled in the art that various changes in form and details may be made therein without departing from the scope and spirit of the invention encompassed by the appended claims.

What is claimed is:

1. A composition comprising at least three of the group consisting of: a) hydroxytyrosol, b) resveratrol, c) lycopene, and d) flavanols or flavonoids.

2. A composition of claim 1 comprising hydroxytyrosol, resveratrol, and lycopene.

3. The composition of claim 2 comprising 6 mg hydroxytyrosol, 25 mg resveratrol, and 1.65 mg lycopene.

4. The composition of claim 2 further comprising at least one of the group consisting of rosemary extract, oregano extract, apple cider vinegar powder, grape seed extract, broccoli juice concentrate, carrot juice concentrate, tomato juice concentrate, beet juice concentrate, spinach juice concentrate, cucumber juice concentrate, brussel sprout juice concentrate, cabbage juice concentrate, celery juice concentrate, kale juice concentrate, asparagus juice concentrate, green bell pepper juice concentrate, cauliflower juice concentrate, parsley juice concentrate, and wheat grass juice concentrate.

5. The composition of claim 1 comprising hydroxytyrosol, resveratrol, lycopene, and flavanols.

6. The composition of claim 5, wherein:
a. hydroxytyrosol is about 5-100 mg;
b. resveratrol is about 25-500 mg;
c. lycopene is about 1-50 mg; and
d. flavanols are about 25-2,000 mg.

7. The composition of claim 5, wherein:
a. hydroxytyrosol is about 6 mg;
b. resveratrol is about 25 mg;
c. lycopene is about 1.65 mg; and
d. flavanols are about 250 mg.

8. The composition of claim 5 further comprising at least one of the group consisting of rosemary extract, oregano extract, apple cider vinegar powder, grape seed extract, broccoli juice concentrate, carrot juice concentrate, tomato juice concentrate, beet juice concentrate, spinach juice concentrate, cucumber juice concentrate, brussel sprout juice concentrate, cabbage juice concentrate, celery juice concentrate, kale juice concentrate, asparagus juice concentrate, green bell pepper juice concentrate, cauliflower juice concentrate, parsley juice concentrate, and wheat grass juice concentrate.

9. The composition of claim 5, wherein
a. hydroxytyrosol is isolated, extracted, or concentrated from olive water, olive pulp, olive oil, olive leaf, or a synthetic source;
b. resveratrol is isolated, extracted, or concentrated from Japanese Knotweed, red wine, red grape juice, grapes, peanuts, cocoa, or chocolate or a synthetic source;
c. lycopene is isolated, extracted, or concentrated from tomatoes, microbial production via fermentation, guava, grapefruit, parsley, basil, or persimmons; and
d. flavanols are isolated, extracted, or concentrated from cocoa, chocolate, or tea or a synthetic source.

10. The composition of claim 9, wherein
a. hydroxytyrosol is isolated, extracted, or concentrated from olive water or olive pulp;
b. lycopene is isolated, extracted, or concentrated from tomatoes; and
c. flavanols are isolated, extracted, or concentrated from cocoa or chocolate.

11. The composition of claim 9, wherein:
a. hydroxytyrosol is about 5-100 mg;
b. resveratrol is about 25-500 mg;
c. lycopene is about 1-50 mg; and
d. flavanols are about 25-2,000 mg.

12. The composition of claim 9, wherein:
a. hydroxytyrosol is about 6 mg;
b. resveratrol is about 50 mg;
c. lycopene is about 10 mg; and
d. flavanols are about 250 mg.

13. The composition of claim 1 comprising hydroxytyrosol, resveratrol, lycopene, and flavonoids.

14. The composition of claim 13, wherein:
a. hydroxytyrosol is about 5-100 mg;
b. resveratrol is about 25-500 mg;
c. lycopene is about 1-50 mg; and
d. flavonoids are about 25-2,000 mg.

15. The composition of claim 13, wherein:
a. hydroxytyrosol is about 6 mg;
b. resveratrol is about 25 mg;
c. lycopene is about 1.65 mg; and
d. flavonoids are about 250 mg.

16. The composition of claim 13, wherein:
a. hydroxytyrosol is isolated, extracted, or concentrated from olive water, olive pulp, olive oil, olive leaf, or a synthetic source;
b. resveratrol is isolated, extracted, or concentrated from Japanese Knotweed, red wine, red grape juice, grapes, peanuts, cocoas, or chocolate;
c. lycopene is isolated, extracted, or concentrated from tomatoes, microbial production via fermentation, guava, grapefruit, parsley, basil, or persimmons; and
d. flavonoids are isolated, extracted, or concentrated from acacia tree heartwood (acacia catechu), berries, tree fruits, beans, tree nuts, green vegetables, or red vegetables.

17. The composition of claim 16, wherein;
a. berries are selected from the group consisting of blueberries, cranberries, and cherries;
b. tree fruits are selected from the group consisting of bananas and citrus fruits;
c. beans are selected from the group consisting of black beans and kidney beans;
d. tree nuts are selected from the group consisting of cashews, walnuts and pecans;
e. green vegetables are selected from the group consisting of broccoli and green peppers; and
f. red vegetables are red peppers.

18. A method of improving at least one risk factor in a subject possessing said risk factor or risk factors comprising administering to said subject an effective amount of a composition according to claim 5, wherein
the risk factors are selected from the group consisting of elevated C-reactive protein, hypertension, elevated low-density lipoprotein (LDL) cholesterol levels, low high-density lipoprotein (HDL) cholesterol levels, elevated triglycerides, elevated tumor necrosis factor (TNF), low tissue oxygenation, elevated fasting or post-prandial insulin levels, elevated fasting or post-prandial glucose levels, elevated fasting or post-prandial hemoglobin A1c (HbA1c), elevated non-esterified free fatty acids (NEFAs), elevated body mass index (BMI), hypothyroidism, hyperthyroidism, and impaired cognition;
such that at least one of said risk factors is improved.

19. The method of claim 18, wherein the said subject has not been identified as suffering from one or more of diseases or disorders selected from the group consisting of cardiovascular disease, athlerosclerosis, heart failure, hypercholesterolemia, diabetes, inflammation associated with pain, and metabolic syndrome.

20. A method of treating dyslipidemia in a subject comprising the administration to said subject an effective amount of a composition according to claim 5, such that said dyslipidemia is improved.

21. The method of claim 20, wherein the treatment affects one or more of the following parameters selected from the group consisting of: reducing total cholesterol levels, reducing LDL cholesterol levels, reducing levels of reactive oxygen species (ROS), reducing triglycerides, and increasing HDL cholesterol levels.

22. A method of reducing inflammation or an inflammation-mediated disease or disorder in a subject comprising the administration to said subject an effective amount of a composition according to claim 5, such that said inflammation or inflammation-mediated disease or disorder is improved.

23. The method of claim 22, wherein said subject is identified as having one or more parameters selected from the group consisting of: elevated C-reactive protein, elevated TNF, elevated LDL cholesterol levels, low HDL cholesterol levels, elevated triglycerides, low tissue oxygenation, elevated fasting or post-prandial insulin levels, elevated fasting or post-prandial glucose levels, elevated fasting or post-prandial HbA1c, elevated non-esterified free fatty acids (NEFAs), and elevated body mass index (BMI);
such that at least one of said parameters is improved.

24. The method of claim 22, wherein the inflammation or inflammation-mediated disease or disorder is associated with pain.

25. The method of claim 24, wherein said subject suffers from joint inflammation, reduced range of motion, or joint pain.

26. The method of claim 24, wherein said inflammation-mediated disease or disorder is osteoarthritis or rheumatoid arthritis.

27. The method of claim 22, wherein the treatment reduces levels of ROS in the subject.

28. The method of claim 22, wherein the inflammation or inflammation-mediated disease or disorder is skin inflammation.

29. The method of claim 28, wherein the treatment of said inflammation or inflammation-mediated disease or disorder affects at least one of the parameters selected from the group consisting of: reducing transcutaneous pressure of oxygen, increasing oxygen saturation in the microcirculation (StO2), reducing skin redness or skin irritation, and reducing ROS.

30. The method of claim 28, wherein the inflammation-mediated disease or disorder is eczema or psoriasis.

31. A method of treating hyperglycemia in a subject identified as suffering from hyperglycemia comprising the administration to said subject an effective amount of a composition according to claim 5, such that said hyperglycemia is improved.

32. The method of claim 31, wherein the hyperglycemia is identified in the subject using fasted or post-prandial glucose measurements.

33. A method of treating hyperinsulinemia in a subject identified as suffering from hyperinsulinemia comprising the administration to said subject an effective amount of a composition according to claim 5, such that said hyperinsulinemia is improved.

34. The method of claim 33, wherein the hyperinsulinemia is identified in the subject using fasted or post-prandial insulin measurements.

35. A method of reducing elevated HbA1c levels in a subject identified as having elevated HbA1c levels comprising the administration to said subject an effective amount of a composition according to claim 5, such that said elevated HbA1c levels are reduced.

36. The method of claim 35, wherein the elevated HbA1c levels is identified in the subject using fasted or post-prandial HbA1c measurements.

37. A method of treating diabetes in a subject comprising the administration to said subject an effective amount of a composition according to claim 5.

38. The method of claim 37, wherein the treatment reduces one or more parameters selected from the group consisting of glucose levels, insulin levels, triglycerides, NEFAs, ROS levels, and HbA1c.

39. A method of treating cardiovascular disease in a subject comprising the administration to said subject an effective amount of a composition according to claim 5.

40. The method of claim 39, wherein the treatment reduces one or more parameters selected from the group consisting of blood pressure, athlerosclerosis, platelet aggregation, total cholesterol levels, ROS levels, C-reactive protein, TNF, BMI, triglycerides, and LDL cholesterol levels.

41. A method of reducing platelet aggregation in a subject comprising the administration to said subject an effective amount of a composition according to claim 5.

42. A method of reducing ROS in a subject comprising the administration to said subject an effective amount of a composition according to claim 5.

43. The method of claim 42, wherein the ROS is oxygen radical, superoxide, or singlet oxygen.

44. A method of increasing total antioxidant capacity in a subject comprising the administration to said subject an effective amount of a composition according to claim 5.

45. A method of treating aging or a disease or disorder associated with aging in a subject comprising the administration to said subject an effective amount of a composition according to claim 5.

46. The method of claim 45, wherein the aging or the disease or disorder associated with aging is mediated through one or more of sirtuin pathways.

47. The method of claim 46, wherein the sirtuin pathway is Sirt1 or Sirt3.

48. The method of claim 45, wherein the aging or the disease or disorder associated with aging is mediated through ROS.

49. The method of claim 48, wherein the ROS is oxygen radical, superoxide, or singlet oxygen.

50. A method of treating a cognitive disorder in a subject comprising the administration to said subject an effective amount of a composition according to claim 5, such that said cognitive disorder is improved.

51. The method of claim 50, wherein the cognitive disorder is impaired memory, dementia, or Alzheimer's disease.

52. A method of treating a subject at risk of developing diabetes, said method comprising:
a. identifying said subject as at risk of developing diabetes; and
b. administering to said subject a composition according to claim 5.

53. The method of claim 52, wherein the subject is identified at risk of developing diabetes through one or more of parameters selected from the group consisting of elevated glucose levels, elevated insulin levels, elevated HbA1c levels, elevated NEFAs, elevated triglycerides, and elevated BMI.

54. The method of claim 53, wherein the elevated glucose levels, elevated insulin levels, elevated HbA1c levels, elevated NEFAs, or elevated triglycerides is measured in a fasted state.

55. The method of claim 53, wherein the elevated glucose levels, elevated insulin levels, elevated HbA1c levels, elevated NEFAs, or elevated triglycerides is measured in a post-prandial state.

56. A method of treating a subject at risk of developing cardiovascular disease, said method comprising:
a. identifying said subject as at risk of developing cardiovascular disease; and
b. administering to said subject an effective amount of a composition according to claim 5.

57. The method of claim 56, wherein the subject is identified at risk of developing cardiovascular disease through one or more of the parameters selected from the group consisting of hypertension, elevated total cholesterol levels, elevated LDL cholesterol levels, low HDL cholesterol levels, elevated C-reactive protein levels, elevated TNF levels, elevated triglycerides, elevated BMI, and atherosclerosis.

58. A method of treating obesity in a subject suffering from said disease comprising the administration to said subject an effective amount of a composition according to claim 5, such that said obesity is improved.

59. The method of claim 58, wherein said treatment affects one or more of the following parameters selected from the group consisting of: reducing body weight, reducing percent body fat, reducing BMI, reducing fasting glucose levels, reducing post-prandial glucose levels, reducing fasting insulin levels, reducing post-prandial insulin levels, and reducing ROS levels.

60. A method of treating or preventing skin inflammation due to sun exposure in a subject comprising the administration to said subject an effective amount of a composition according to claim 5.

61. The method of claim 60, wherein a) recovery time from sunburn or b) ROS is reduced.

62. A method of treating an allergic reaction in a subject comprising the administration to said subject an effective amount of a composition according to claim 5, such that said allergic reaction is reduced.

63. The method of claim 62, wherein the allergic reaction is due to a food allergy.

64. The method of claim 62 or 63, wherein the treatment reduces at least of one of the following parameters selected from the group consisting of: a) blood antibodies in response to said allergy, b) eosinophil levels, and c) ROS.

65. A method of enhancing tissue oxygenation in a subject comprising the administration to said subject an effective amount of a composition according to claim 5.

66. The method of claim 65, wherein enhancing tissue oxygenation affects at least one of the parameters selected from the group consisting of: a) increasing blood hemoglobin oxygen saturation (e.g., SO2, SaO2), b) decreasing partial pressure of oxygen (SpO2), c) decreasing transcutaneous pressure of oxygen, d) improvement in VO2 measurement, and e) increasing oxygen saturation in the microcirculation (StO2).

67. The method of claim 65, wherein the tissue is skin or muscle.

68. The method of claim 65, wherein the enhanced tissue oxygenation improves fitness level or exercise performance in said subject.

69. A method of treating hyperthyroidism in a subject comprising the administration to said subject an effective amount of a composition according to claim 5, such that said hyperthyroidism is improved.

70. A method of treating hypothyroidism in a subject comprising the administration to said subject an effective amount of a composition according to claim 5, such that said hypothyroidism is improved.

71. A method of enhancing athletic performance in a subject comprising the administration to said subject an effective amount of a composition according to claim 5.

72. The method of claim 71, wherein the method improves walking speed, running speed, strength, endurance, or VO2 measurement.
